(19) United States
(12) Patent Application Publication
(10) Pub. No.: US 2007/0027430 A1
(43) Pub. Date: Feb. 1, 2007

(54) Trigger-activatable injection device

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/454,148

(22) Filed: Jun. 15, 2006

Related U.S. Application Data
(63) Continuation of application No. PCT/CH04/00715, filed on Nov. 29, 2004.

(30) Foreign Application Priority Data
Dec. 18, 2003 (CH) 2189/03
Dec. 18, 2003 (DE) 203 19 648.1

(51) Int. Cl. A61M 5/00

(52) U.S. Cl. 604/207; 604/187

(57) ABSTRACT

An injection device including a housing formed by two housing parts moveable in relation to each other, a drive element urged by a spring in an injection direction and releaseably held against movement, and a release element moveable from a lock position to an intermediate position due to the movement of the housing parts, the drive element remaining held against movement when the release element is in the intermediate position, the release element further moveable from the intermediate position to a release position in which the drive element is released.

### CROSS-REFERENCE TO RELATED APPLICATION(S)

This application is a continuation of International Application PCT/CH2004/000715, filed on Nov. 29, 2004, which claims priority to Swiss Application CH 2189/03, filed on Dec. 18, 2003 and German Application DE 203 19 648.1, filed on Dec. 18, 2003, the entire contents of all of which are incorporated herein by reference.

### BACKGROUND

The present invention relates to devices for delivering, administering, injecting or dispensing substances, and methods of making and using such devices. More particularly, it relates to an injection device for administering an injectable product, such as a liquid medicament such as insulin, a growth hormone, heparin, an osteoporosis preparation, etc. In some preferred embodiments, the injection device is an injection pen or an automatic injector.

Injection devices of numerous designs are known. They are generally used for administering or delivering medicinal substances or medicaments, and, in many cases, are used by a patient to self-administer a substance.

Automatic injection devices are one type of injection devices. Many automatic injectors have a release lock intended to prevent an injection procedure from being inadvertently triggered. An example of an automatic injector is described in document DE 4037418. This automatic injector has a housing for accommodating a container of active substance. The housing consists of two parts which can be displaced relative to one another from a first and a second position along an axis. The automatic injector also has a release mechanism, a dispensing mechanism for dispensing the active substance which is held by means of the release mechanism in a position into which it is urged or held by a spring, and a trigger device in the form of a push button, which can likewise be operated in the direction of said axis. In this instance, one housing part accommodates the trigger device and the other housing part accommodates the release mechanism. When the two housing parts are in the first position relative to one another, the release mechanism is disposed at a greater distance from the trigger device than in it is in the second position and operating the trigger device has no effect. When the housing parts are in the second position, the trigger device is closer to the release mechanism and is therefore in a position in which it can cause the release of the dispensing mechanism.

### SUMMARY

An object of the present invention is to provide an injection device which offers a high degree of safety against inadvertent triggering while requiring only a few components.

In one embodiment, the present invention comprises an injection device comprising a housing formed by two housing parts moveable in relation to each other, a drive element urged by a spring in an injection direction and releaseably held against movement, and a release element moveable from a lock position to an intermediate position due to the movement of the housing parts, the drive element remaining held against movement when the release element is in the intermediate position, the release element further moveable from the intermediate position to a release position in which the drive element is released.

In one embodiment, the present invention comprises a trigger-activatable injection device comprising a housing portion, a reservoir for an injectable product, an injection needle with a needle tip at one end, a needle guard moveable relative to the housing portion and to the injection needle from a protective position generally surrounding the needle to a retracted position, a drive mechanism for powering an injection, the drive mechanism releaseably held in a retaining engagement to prevent a driving movement, a switching element coupled to the needle guard so that the movement of the needle guard to the retracted position causes the switching element to move from a locked position to a coupled position, and an activatable trigger element for triggering the drive mechanism which, upon activation, is coupled with the switching element when the switching element assumes the coupled position and causes a movement of the switching element from the coupled position to a release position, wherein the retaining engagement of the drive mechanism is not released until the switching element is in the release position.

In one embodiment, the present invention comprises an injection device comprising a housing portion, a reservoir for the product to be injected, namely the active ingredient, an injection needle, a needle guard, a drive mechanism for the injection and a trigger mechanism for triggering the drive mechanism. The housing portion, or another housing portion of the injection device connected to it, directly constitutes the reservoir. In some preferred embodiments, the reservoir is a container accommodated in the housing portion or the other housing portion. The injection device has a reservoir holder, which holds and centers the reservoir and is itself held and centered in a housing portion. The injection needle may be attached to the reservoir or to the reservoir holder. Accordingly, it projects axially forward, from the reservoir or the reservoir holder, and has a needle tip at its front, or distal end. The needle guard is joined to the housing portion so that it is able to move relative to the housing portion and relative to the injection needle from a protective position in which the injection needle is surrounded to a point beyond its needle tip, backwards, or proximally, axially relative to the housing portion and to the injection needle as far as a retracted position. The needle guard may be a purely visual guard which can be moved proximally against an elastic return force and pushed back in the distal direction again when the return force is released. Alternatively, however, it may also be locked in the protective position to prevent a return movement, in which case the locking action must be released prior to the injection, after which it may be releasable or non-releasable.

The drive mechanism may comprise a dispensing drive with a conveyor element or drive element and a dispensing drive for the drive element, and the drive element acts on the product disposed in the reservoir in order to dispense the product. In preferred embodiments in which the injection device is an automatic injector, the drive mechanism not only has such a dispensing drive but also an injection drive for the injection needle. This being the case, the same force generator may be used to drive the dispensing drive and the injection drive. For example, a mechanical spring, e.g., a coil spring pre-stressed to a specific pressure, serves as the injection drive as well as the dispensing drive. However, separate force generators could also be provided for each of the two drives, for example a coil spring pre-stressed to a specific pressure. In some preferred embodiments, the injection drive is a force generator which assumes an elastically tensed state prior to an injection, from which it drives the injection needle so as to inject it in and/or through the skin in the distal, i.e. forward drive, direction. The drive mechanism may be designed so that it effects only a driving movement causing the dispensing action or a driving movement causing dispensing and prior injection, or it may effect one driving movement for initial injection and another driving movement for the dispensing process.

When the injection device is in an initial state, the drive mechanism is held in a releasable retaining engagement so that it can not effect any driving movement and can not effect a driving movement causing the injection needle to be injected. If the injection device is not an automatic injector, but an injector with an injection needle by means of which the user must inject himself, only a dispensing drive is provided and it is on this which the retaining engagement therefore acts.

In some embodiments, to trigger the drive mechanism, a moving housing part or the needle guard, trigger member or trigger element and a release element, e.g., a switching element, co-operate with one another. For example, the switching element and the needle guard may be coupled with one another so that the movement of the needle guard out of the protective position into the retracted position causes a movement of the switching element out of a latching or locking position into a coupled or intermediate position. Once the switching element assumes the coupled position, the trigger element can be moved into a coupled engagement with the switching element by operating the trigger element. In the coupled engagement, the triggering movement of the trigger element caused by activation is converted into a movement of the switching element out of the coupled position into a released position. As long as the switching element has not assumed the released position, it prevents the retaining engagement of the drive mechanism from being released. The retaining engagement exists directly, or via one or more intermediate elements, between the drive mechanism and the housing portion, which means that the drive mechanism is not able to effect any driving movement relative to the housing portion in the retained engagement. The driving movement is or includes a axial linear movement of the drive mechanism in a forward drive direction, for example in the distal direction. If the injection device is an automatic injector, the drive mechanism may be coupled with the injection needle so that it drives the injection needle in the drive direction, i.e. in the direction of its own driving movement.

The switching element guides the movement from the locking position via the coupled position as far as the released position as a unit. As a result, the number of parts needed for the trigger system is kept low. The switching element may be made as a single piece, but may also be an assembled structure in principle, although the movement from the locked position as far as the released position is effected as a unit as if it were a single stiff body, at least as far as this movement is concerned.

The movement of the switching element from the locked position into the released position is a movement in a single direction only, e.g., an axial movement in the proximal direction.

The trigger element projects out from the housing portion so that it can be operated directly. In some embodiments, it is preferably a trigger button, i.e., a push button. The movement effected when the trigger element is operated is a movement transversely to a proximal-distal axis of the injection device.

The coupled engagement between the trigger element and the switching element may be obtained on the basis of a sliding pressure or frictional contact. At least one of the switching element and trigger element acts as a switching cam and the other an engaging element sliding on the switching cam. The sliding pressure contact is established due to the trigger movement of the trigger element when the switching element assumes the coupled position. In the sliding pressure contact, the trigger element pushes the switching element out of the coupled position into the released position.

In the positions which the switching element assumes during an injection prior to assuming the released position, it locks a blocking mechanism securing the retaining engagement between the drive mechanism and the housing portion, preventing the retaining engagement from being released. The blocking mechanism is elastically biased or urged towards a release of the retaining engagement but is prevented from releasing the engagement by the switching element. The switching element pushes a blocking element of the blocking mechanism into the retaining engagement. The switching member is designed so that it allows the blocking element to snap out of the retaining engagement in the released position, thereby automatically releasing the retaining engagement.

In one embodiment of the present invention in the form of an automatic injector, the trigger element or trigger member can be moved in a plane disposed transversely to the longitudinal axis of the automatic injector and the release element can be moved parallel to the longitudinal axis of the automatic injector. The release element may, for example, be moved from the locked position into the intermediate position by moving the housing parts or portions relative to one another. In the intermediate position, the switching element in the form of a release element may co-operate with a locking device such as an injection blocking element, which secures the drive element and the forward-drive structure in a locked position. When the release element is in the intermediate position, it can be moved farther into the released position in which the drive element is released by means of the release element. To this end, the release can have a guide mechanism which co-operates with the release element so that it is moved into the released position. The guide mechanism may, for example, be in the form of a surface disposed at an angle relative to the longitudinal axis of the automatic injector on the release element, e.g., on the trigger element. When the trigger element is operated, an edge of the release element is moved so that it lies on the oblique surface of the trigger element and is pushed along the oblique surface into the released position when the trigger element is activated.

As long as the release element is in the locked position, operating the release element will not trigger the automatic injector. The release element may be biased into a position projecting out of the automatic injector by means of a spring. With this type of embodiment, the automatic injector is triggered in two independent operating steps which are uncoupled form one another. It is therefore not possible to trigger the automatic injector inadvertently.

The locking and unlocking mechanism and the trigger element do not move relative to one another in the longitudinal direction. They move in a plane disposed transversely to the longitudinal axis of the automatic injector, as a result of which no additional room for movement has to be provided for these components in the longitudinal direction of the automatic injector. This means that the automatic injector can be made shorter overall.

In another embodiment of the present invention, the trigger element can be moved in the longitudinal direction of the automatic injector. This makes it easy to operate the trigger mechanism at one end of the automatic injector. In this embodiment, the release element, in its locked position, prevents the trigger element from moving along it. This ensures that the automatic injector has to be positioned first of all and then the operating element pressed. In one type of embodiment, the release element can be moved in a plane disposed transversely to the longitudinal axis or direction of the automatic injector. Consequently, in one variant, the release element may be guided so that it effects a translating movement from the locked position into the released position. This being the case, the release element has an orifice in the shape of a keyhole and an annular groove is provided on the drive element, in which the slim or narrow part of the keyhole orifice engages when the release element is in the locked position. In another variant, the release element is designed so that it effects a rotating movement from the locked position into the released position. In this instance, the release element has a non-rounded orifice and a head with a non-rounded cross-section is provided on the drive element which does not fit through the orifice except when the release element is in the released position. Both variants require only a few individual parts and enable an inexpensive automatic injector to be provided which is reliable in operation.

### BRIEF DESCRIPTION OF THE DRAWINGS

FIG. 1 illustrates an exemplary embodiment of an automatic injector in accordance with the present invention in an initial state prior to injection,

FIG. 2 shows a needle guard, a switching element and a trigger element of the automatic injector in the initial state,

FIG. 3 shows cross-section A-A indicated in FIG. 1,

FIG. 4 illustrates the automatic injector of FIG. 1 positioned at an injection point prior to triggering,

FIG. 5 shows the needle guard, the switching element and the trigger element of the automatic injector in the state illustrated in FIG. 4,

FIG. 6 shows cross-section B-B indicated in FIG. 4,

FIG. 7 shows the automatic injector of FIG. 1 positioned at an injection point in the triggered state,

FIG. 8 shows the needle guard, the switching element and the trigger element in the triggered state,

FIG. 9 shows cross-section C-C indicated in FIG. 7,

FIG. 10 illustrates a second exemplary embodiment of an automatic injector in accordance with the present invention in an initial state,

FIG. 11 is a longitudinal section through an injection device in accordance with the invention in a state in which it may be supplied, and

FIG. 12, including FIGS. 12a to 12c, show, in cross section, three positions of a release element for the device illustrated in FIG. 11.

### DETAILED DESCRIPTION

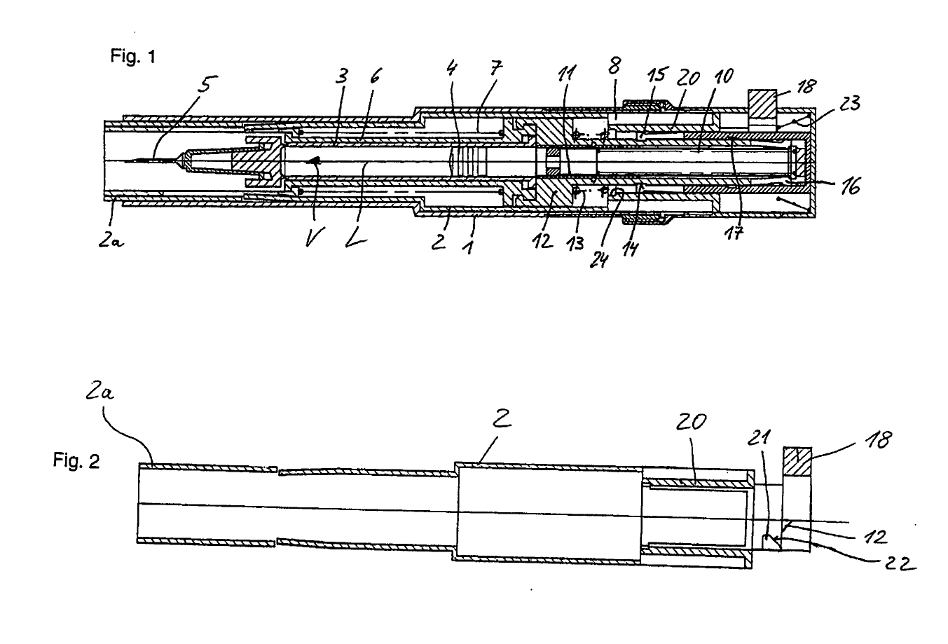

FIG. 1 depicts an exemplary embodiment of an injection device in accordance with the present invention. The depicted embodiment is an automatic injector embodied in the form of an injection pen. It comprises a sleeve-shaped proximal or rear housing portion 1 serving as a gripping piece and a sleeve-shaped distal or front housing portion 2, which forms a needle guard 2a at its distal end. Housing portion 2 is axially guided in housing portion 1, linearly along a common longitudinal axis L.

Housing portion 2 accommodates a reservoir 3, which is filled with an injectable product, for example insulin. The reservoir 3 is a container and, in this example, is a standard ampoule, in which a drive element 4 in the form of a plunger is accommodated. Product is dispensed through an outlet of the reservoir 3 and an injection needle 5 connected to the reservoir 3 by moving the drive element 4 axially in a forward drive direction V. The injection needle 5 is attached to the distal end of the reservoir 3 and its free needle tip projects forward in the forward drive direction V. When the automatic injector is in the initial state illustrated in FIG. 1, the needle guard 2a surrounds the injection needle 5 as far as beyond its needle tip. This corresponds to an axial protective position of the needle guard 2a. The reservoir 3 is held centred in a reservoir holder 6 and is supported by the reservoir holder 6 in the forward drive direction V. Housing portion 2 and hence its needle guard 2a is elastically biased in the forward drive direction V by means of a return spring 7. It can be moved relative to the housing portion 2 against the force of the spring 7 in the direction opposite the forward drive direction V. In the embodiment illustrated as an example here, the spring 7 is directly supported on the housing portion 2 in the forward drive direction V and directly on the reservoir holder 6 in the direction opposite the forward drive direction V.

A drive mechanism is accommodated in the housing portion 1 and is mounted or carried so as to be axially displaceable. The drive mechanism comprises a dispensing drive for dispensing the product and an injection drive for initially injecting the injection needle 5. A dispensing spring 10 forms the dispensing drive. The dispensing spring 10 acts via a dispensing structure 11, which is provided in the form of a dispensing sleeve for driving the product onto the drive element 4. Due to the design of the drive element 4 in the form of a translating plunger, the dispensing structure 11 is a plunger rod. The dispensing spring 10 biases the dispensing structure 11 in the forward drive direction V. When the automatic injector is in the initial state, however, the dispensing structure 11 is in a retaining engagement which prevents the dispensing structure 11 from being moved forwards.

The injection drive is also provided in the form of a spring, namely injection spring 13. When the automatic injector is in the initial state, the injection spring 13 is also biased in the forward drive direction V. Due to its elastic force, it applies pressure to a forward drive structure 12, which is held in a retaining engagement against the force of the injection spring 13 and is prevented from effecting a forward drive movement relative to the housing portion 1. The forward drive structure 12 surrounds the dispensing structure 11, providing a mount and linear guide for the latter in the axial direction. Both the dispensing spring 10 and the injection spring 13 are supported on the housing portion 1 in the direction opposite the forward drive direction V, the injection spring being directly supported on a bearing block 8 which is not able to move axially and radially and which does not turn, and in this sense is fixedly connected to the housing portion 1. The bearing structure 8 also forms an axial linear guide for the forward drive structure 12.

The retaining engagement of the forward drive structure 12 comprises an injection blocking element 15 in the form of an elastic snapper and a projection, by means of which it projects into a recess 14 provided on an external surface of the sleeve-shaped forward drive structure 12. The injection blocking element 15 is not able to move axially relative to the housing portion 1. Several of the injection blocking elements 15 are disposed around the periphery of a sleeve body, each in the form of axial resilient tongues.

To provide a retaining engagement for the dispensing structure 11, the forward drive structure 12 also has several resilient tongues on its proximal end, which act as dispensing blocking elements 16 by each forming a forward drive stop for the dispensing structure 11. The same sleeve body forming the injection blocking elements 15 pushes the dispensing blocking elements 16 radially inwards into the retaining engagement with the dispensing structure 11.

The injection blocking elements 15 are pushed radially inwards by means of a switching element 20 into the retaining engagement, i.e. into the recesses 14 or the recess 14 provided in the forward drive structure 12 in the form of a peripheral groove. To this end, the switching element 20 has a sleeve portion surrounding the forward drive structure 12 and the injection blocking elements 15 and pushes them into the recesses 15 or the peripherally extending recess 15. The bearing structure 8 provides a mount for the switching element 20 so that it can be axially displaced. The bearing structure 8 and/or the sleeve body forming injection blocking elements 15 guide the switching element 20 linearly in the axial direction. The switching element 20 is in an axial pressure contact with the housing portion 2, causing a movement of the housing portion 2 in the direction opposite the forward drive direction V and an identical movement of the switching element 20. Finally, the switching element 20 is supported on the housing portion 1 by means of a return spring 23 in the direction opposite the forward drive direction V. In the initial state, the switching element 20 assumes an axial locking position in which it locks the injection blocking elements 15 in their retaining engagement with the forward drive structure 12.

In a proximal portion of the housing portion 1, a trigger element 18 projects radially outwards from its external surface. The trigger element 18 can be pushed deeper into the housing portion 1, e.g., by applying radial pressure, and, due to such an operation, moves in a direction disposed transversely, in this embodiment radially, with respect to a mid-longitudinal axis L of the automatic injector pointing in the forward drive direction V.

FIG. 2 shows the housing portion 2 with the needle guard 2a, the switching element 20 and trigger element 18 released from the automatic injector illustrated in FIG. 1 in the relative positions which they assume when the automatic injector is in the initial state. Only the axial pressure contact exists between the housing portion 2 and the switching element 20. The trigger element 18 and the switching element 20 are not engaged. As illustrated, however, a switching cam 19 is provided, which is inclined with respect to the forward drive direction V and with respect to the direction of the transverse movement of the trigger element 18. The forward drive direction V and the direction of transverse movement, i.e. the direction of the triggering movement of the trigger element 18, are oriented perpendicular to one another. The switching cam 19 points in the direction opposite the forward drive direction V. The switching element 20 is provided with a cam 21, which forms an engaging element in a coupled engagement of the switching element 20 with the trigger element 18 and slides on the switching cam 19 as the trigger element 18 effects its triggering movement. The switching element 20 is also provided with a switching cam 22, which in the embodiment illustrated as an example is provided on a rear surface of the cam 21. The switching cam 22 may not fulfil any function in terms of triggering the automatic injector. It merely corrects the position of the trigger element 18 in the event that it is inadvertently pushed when the automatic injector is in the initial state. The two switching cams 19 and 22 extend in a straight line, i.e. are oblique. In principle, however, a non-linear contour would also be feasible.

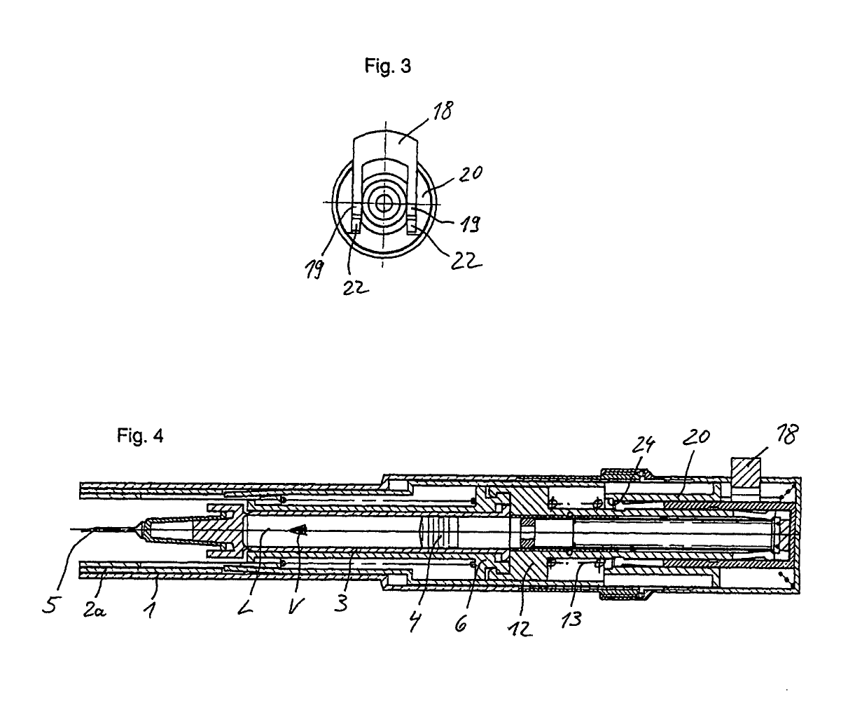

FIG. 3 specifically illustrates the trigger element 18 and the switching element 20 as well as their switching cams 19 and 22 through a cross-section A-A indicated in FIG. 1.

Once the automatic injector has been positioned on or at the desired injection site on the skin, a first step of the injection involves applying pressure against the injection point so that the needle guard 2a and thus the entire housing portion 2 is moved proximally relative to the housing portion 1, i.e. deeper into the housing portion 1, until the distal end of the needle guard 2a and the distal end of the housing portion 1 are disposed at the same axial height.

FIG. 4 illustrates the automatic injector at the end of this first phase of the injection. Due to the rearward movement of the needle guard 2a into the retracted position illustrated in FIG. 4, in which the needle tip sits directly above the skin, the needle guard 2a and the housing portion 2 have moved the switching element 20 back by the distance of their own rearward movement relative to the housing portion 1 and in particular relative to the trigger element 18.

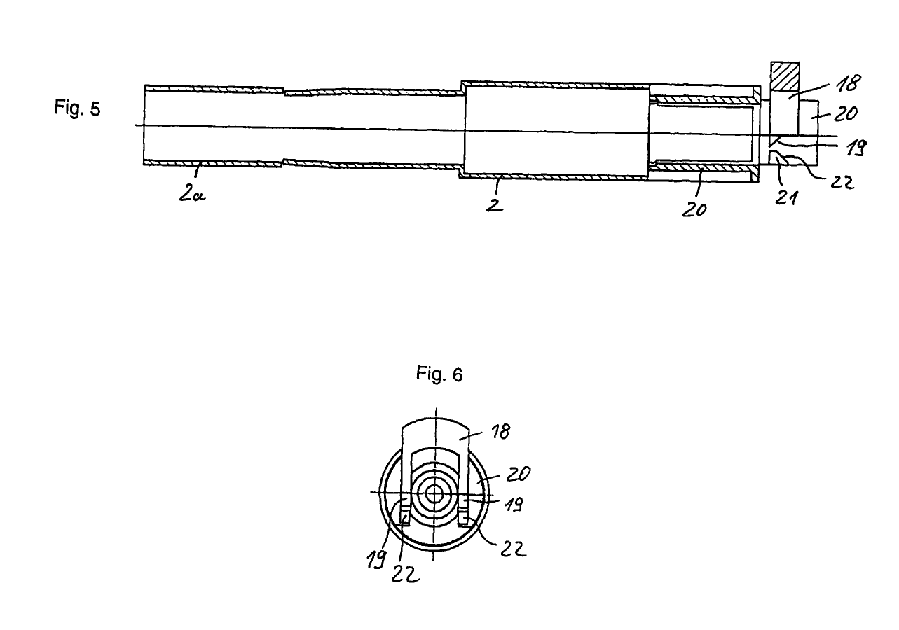

FIG. 5 illustrates the relative position which the housing portion 2, switching element 20 and trigger element 18 have now assumed. The axial position of the switching element 20 relative to the trigger element 18 is such that the switching cam 19 has effected a triggering movement of the trigger element 18, i.e. when the trigger element 18 was depressed, to make a pressure sliding contact with the cam 21 and slides along the cam 21 in the pressure sliding contact. The axial position now assumed by the switching element 20 is therefore referred to as the coupled position.

When the switching element 20 is in the coupled position and the injection is triggered by pressing the trigger element 18, the latter moves relative to the housing portion 1 and in particular relative to the switching element 20 towards its cam 21. It moves into said sliding pressure contact with the cam 21 and, because of the inclined contour of the switching cam 19, pushes the cam 21 and thus with it the switching element 20 in the direction opposite the forward drive direction V, i.e. it pulls the switching element 20 in the proximal direction. The cam 21 forms an engagement member for this axial movement of the switching element 20, which slides along the switching cam 19 when the trigger element 18 is pushed in. Since the trigger element 18 is guided and effects only the radial triggering movement but is unable to move axially relative to the housing portion 1, the switching element 20 is pulled farther back in the direction opposite the forward drive direction V by means of its cam 21 into an axial end position.

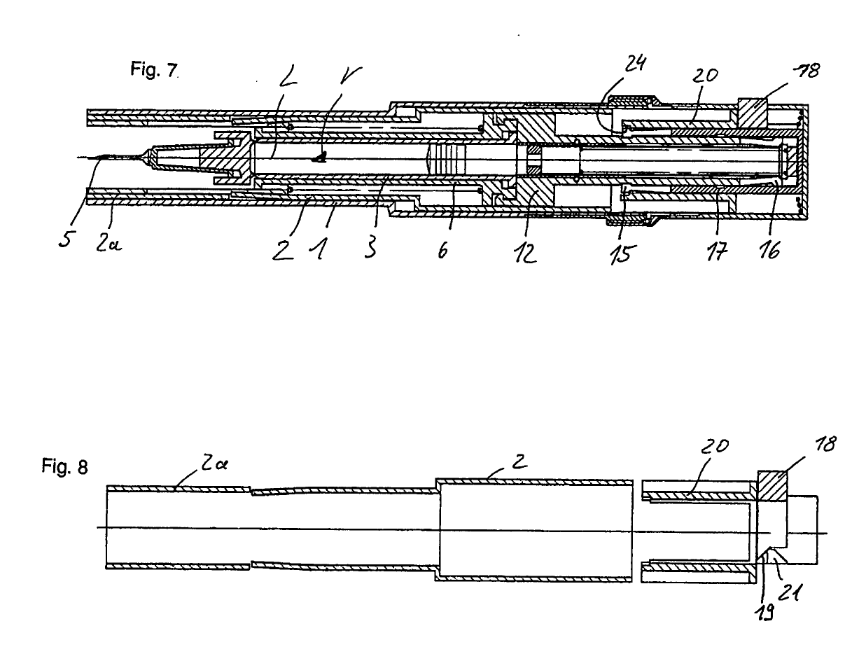

FIG. 7 illustrates the exemplary automatic injector after triggering, i.e. the trigger element 18 has effected its triggering movement and the switching element 20 has assumed its proximal end position relative to the trigger element 18 and relative to the injection blocking elements 15. In its sleeve portion, by means of which it has so far pushed the injection blocking elements 15 into the retaining engagement with the forward drive structure 12, the switching element 20 has a recess 24. In the embodiment illustrated as an example, this is provided in the form of a wider region extending round the internal surface of the sleeve portion of the switching element 20, terminating the switching element at the distal end. When the switching element 20 is in the proximal end position, the injection blocking elements 15 snap radially outwards, out of the retaining engagement with the forward drive structure 12 into the recess 24 due to their natural elastic rebound forces. The retaining engagement of the forward drive structure 12 is therefore released and the forward drive structure 20 is driven forwards in the forward drive direction V by the tensed injection spring 13. As it is driven forwards, it pushes against the reservoir holder 6, which is moved together with the reservoir 3 accommodated in it in the forward drive direction V. During the forward driving movement, the injection needle 5 is moved forwards out of the housing portions 1 and 2 and into and through the skin. The forward driving movement of the forward drive structure 12 is restricted by a stop.

As soon as the forward driving movement of the forward drive structure 12 and, thus, the forward injection movement of the injection needle 5 has ended, the dispensing blocking elements 16 move into a recess 17 of the sleeve body, which also forms injection blocking elements 15, and snap into the recess 17. Due to the forward snapping action of the dispensing blocking elements 16, the retaining engagement of the dispensing structure 11 is released and it now slides relative to the forward drive structure 12 in the forward drive direction V. The dispensing structure 11 thus comes into contact with the drive element 4 and pushes it forwards in the reservoir 3 towards its outlet. As a result, the product is dispensed from the reservoir 3 and through the injection needle 5 and is administered.

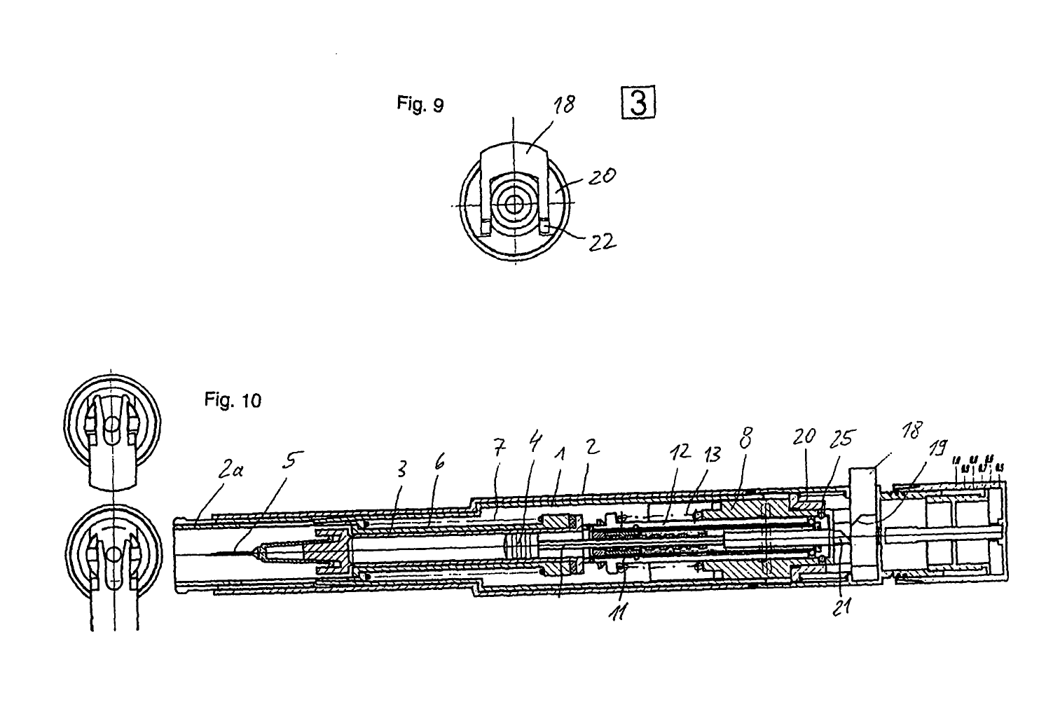

FIG. 8 provides another detailed illustration of the housing portion 2, the switching element 20 and the trigger element 18 in the axial positions which they assume relative to one another after triggering. FIG. 9 illustrates the same state but in a cross-section through C-C indicated in FIG. 7.

FIG. 10 depicts a second exemplary embodiment of an injection device. The automatic injector of the second embodiment differs from the embodiment described as a first example in terms of its injection blocking elements, which are denoted by reference 25 in the embodiment illustrated as a second example. The blocking elements 25 are ball bearings or cylindrical pins, which are also biased radially outwardly and are locked by the switching element 20 to prevent them from moving out of the retaining engagement. To this extent, the switching element 20 of the second embodiment corresponds to that of the embodiment described as a first example. There are also no differences as regards the co-operation with the needle guard 2a and the trigger element 18. The differences relate to another aspect and have nothing to do with the trigger mechanism formed by the needle guard 2a or housing portion 2, the switching element 20 and the trigger element 18. In this respect, the explanations given with reference to the first embodiment apply.

The trigger element 18 is displaceable in a plane disposed transversely to the longitudinal direction of the automatic injector and the release element 20 is displaceable in the longitudinal direction of the automatic injector. The release element 20 is provided in the form of a sleeve, which is axially displaceable relative to the front, distal and rear proximal housing parts 1 and 2 and is disposed inside these housing parts. The trigger element 18 is formed by a button projecting out from the side of the automatic injector. In FIGS. 1 and 2, the release element 20 is in the locked position or protective position, i.e. in a position at a distance from the trigger element 18 and is biased forwards in this locking position by means the cone spring 23. This being the case, the release element 20 co-operates with a locking mechanism in the form of two oppositely lying locking arms 15 which are biased and engage in a recess 14 on the drive element 12, thereby locking it so that it can not be pushed forwards. Naturally, it would also be possible to provide only one locking arm or more than two locking arms. The locking arms 15 are biased forward by means of the internal surface of the sleeve of the release element 20 into the recesses 14 of the drive element 12. The locking mechanism could also be provided in the form of ball bearings or a forward-biased annular element, for example, co-operating with the drive element in a locking relationship.

In FIGS. 4 and 5, the release element 20 is shown in an intermediate or coupled position. The front housing part 2 is pushed inside the automatic injector relative to the rear housing part 1, for example when placed on the injection site on the skin of a patient. The release element 20 adjoins the front housing part 2 and is pushed together with it relative to the rear housing part 3 into the intermediate position. The locking arms 15 slide along the internal surface of the sleeve of the release element 20 and are pushed ever further into the recesses on the drive element 12 by the sleeve of the release element 20 so that they prevent said drive element 12 from moving forwards. When the automatic injector is removed from the injection site, the release element 20 is pushed backwards by the cone spring 23 back into the locked position.

The release element 20 has reached the intermediate position within the movement range of the trigger element 18 and can now only co-operate with a guide mechanism on the trigger element 18 and be moved by the latter into a released position. In the embodiment illustrated as an example here, the guide mechanism is provided in the form of the surface 22 on the trigger element 18 extending at an angle with respect to the longitudinal axis of the automatic injector, forming a switching cam. Alternatively, a similar surface or a guide mechanism could also be provided on the release element 20. Provided on the sleeve of the release element 20 is an extension in the direction of the trigger element 18, which has a lug or a cam 21 which, when the oblique surface 22 of the trigger element 18 is in the immediate position, moves in a radial direction towards the longitudinal axis of the automatic injector, as may best be seen from FIG. 5.

FIGS. 7 and 8 illustrate the release element 20 in a released position. When the release element 20 is in the intermediate position, the trigger element 18 may interact with the release element 20. When the trigger element 18 is operated, the oblique surface 22 is moved toward the lug 21 of the release element 20. The lug 21 moves so that it lies on the oblique surface 22 and, as the trigger element 18 is pushed farther in, slides along the oblique surface so that the release element 20 is pulled or pushed in the direction of the trigger element 18 due to the angled geometry until the release element 20 is in the released position. In the released position, a region with a wider cross-section of the sleeve of the release element 20 moves so that it lies opposite the locking arms 15 so that they move radially outwards out of the recesses 14 of the drive element 12 due to their forward bias and release the drive element 12. The drive element can now act on the container 3 of active substance and the injection needle 5 and push them out beyond the front end of the housing parts 1 and 2, resulting in an injection at the injection site.

In this embodiment, triggering can only take place if the front sleeve 2 is pushed back into the rear sleeve 1 and the trigger button 18 is simultaneously depressed.

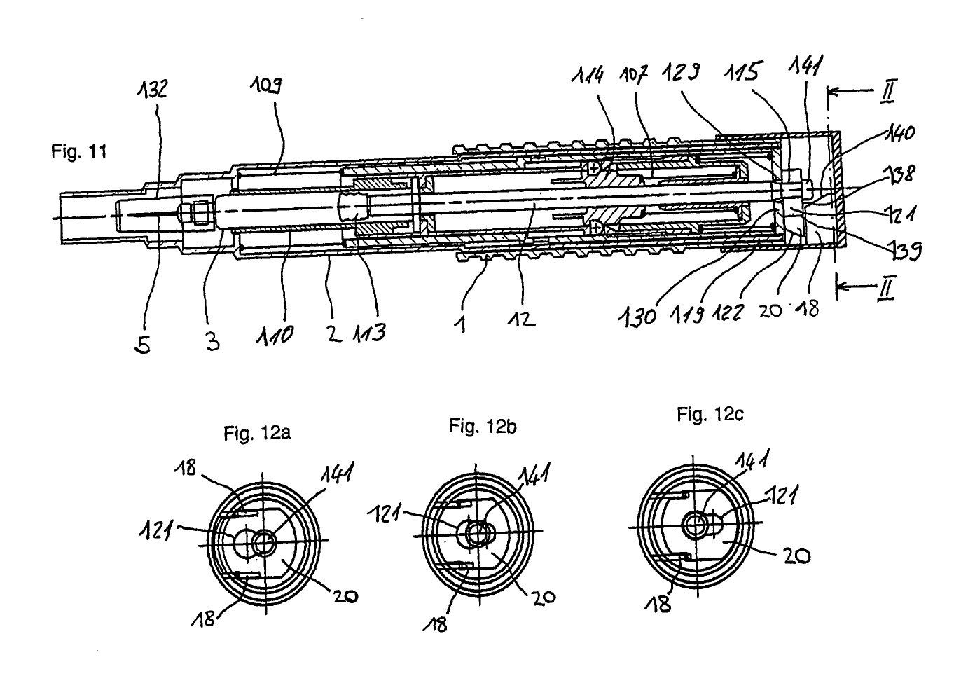

FIGS. 11 and 12a to 12c illustrate another embodiment of an injection device in accordance with the present invention.

The injector illustrated in longitudinal section in FIG. 11 is designed for one-off use only and has a housing comprising a rear housing part 1 and a front housing part 2, which can be moved towards one another from the position illustrated in FIG. 11 against the force of a spring 109. A container 3 filled with an active substance is accommodated in a housing sleeve 110 in the interior of the automatic injector. At its front end, the container 3 supports an injection needle 5 and can be displaced axially in the housing in conjunction with the housing sleeve 110 by the force of a drive spring 107 in order to inject the injection needle 5 in the skin of a patient. The injection needle 5 is not essential to the invention and the trigger mechanism described here may also be used with a needle-less automatic injector. A forward drive structure comprising a rod-shaped drive element 12 engages by means of its front end facing the injection needle 5 in the container 3, where it is connected to a plunger 4, the purpose of which is to dispense the active substance contained in the container 3. Between the two ends of the drive element 12, the latter is connected to a transmission part 114, by means of which the force of the drive spring 107 is transmitted to the drive element 12. At its rear end remote from the injection needle 5, the drive element 12 has an annular groove 115, the purpose of which will be described below.

The injector is illustrated in the tensed and ready-to-use state in FIG. 11. Biased forwards in the direction towards the container 3 by the force of the compressed drive spring 107, the drive element 12 is locked at its rear end by a release element 20, which engages in the annular groove 115. The release element 20 sits on a retaining plate 130 and is able to move transversely to the longitudinal axis of the automatic injector, against the force of a spring, although this is not illustrated. Accordingly, the drive element 12 projects with its rear end through an orifice 129 in this retaining plate 130. A trigger element 18 is provided in the form of a cover, which fits onto the rear end of the automatic injector and which can be activated in its axial direction, against the force of a spring, although this is not illustrated. As clearly illustrated in FIG. 11, the trigger element 18 can not be operated when the automatic injector is in the illustrated operating position because it sits against the flat surface 138 of the release element 20.

In order to use the injector, the needle guard cap 132 which keeps the injection needle 5 sterile must first be removed. The automatic injector is then gripped by the rear housing part 1, placed so that the front housing part 2 sits on the skin of the patient and pushed lightly, whereupon the front housing part 2 pushes against the force of the spring 109 relative to the rear housing part 1. As this happens, a tongue 119 formed on the front housing part 2 moves into abutment with an oblique surface 122 of the release element 20 and moves the release element transversely to the longitudinal direction of the automatic injector into an intermediate position. The distance covered by the release element 20 is not enough to release the drive element 12. However, an edge 139 on the release element 20 moves into the region of an oblique surface 140 provided on the trigger element 18 so that the trigger element 18 can now be activated in the axial direction of the automatic injector. Due to the contact of the oblique surface 140 with the edge 139, the release element is moved further until it reaches its released position, in which it releases the drive element 12, triggering an injection.

FIGS. 12a to 12c illustrate a cross-section along line II-II indicated in FIG. 11 showing how the release element 20 operates. FIG. 12a illustrates the release element 20 in the same position as that shown in FIG. 11, namely in a locked position. An orifice 121 is provided in the release element 20, which has the shape of a keyhole. In the locked position illustrated in FIG. 12a, the narrower part of the orifice 121 engages in the annular groove 115 of the drive element 12 and holds it locked as a result. FIG. 12b illustrates the intermediate position in which the tongue 119 has pushed the release element 20 into its intermediate position. A head 141 of the drive element axially adjoining the annular groove 115 is now disposed at the transition between the narrow part and the wide part of the orifice 121 in the release element, as a result of which the drive element 12 is still held locked in its position biased by the spring 107. Finally, FIG. 12c shows the released position of the release element 20. The wider part of the orifice 121 has been pushed into the region of the annular groove 115 so that the head 141 can now enter the wider part of the orifice 121 and the injection is triggered.

The type of embodiment of the invention described above and illustrated in the drawings is based on a translating movement of the release element 20. In another type of embodiment, the release element 20 can be moved in rotation. In an embodiment of this type, the tongue 119 is bevelled at its end directed towards the release element 20 so that the tongue 119 rotates the release element 20 about the longitudinal axis of the automatic injector when the housing parts 1, 2 are moved relative to one another. In this instance, the oblique surface 140 of the trigger element 18 is not oriented radially as illustrated in FIG. 11 but in the circumferential direction. In this embodiment too, the release element has three positions and is moved by the tongue 119 from the locked position into the intermediate position and by the trigger element 18 from the intermediate position into the released position. The orifice in the release element and the head 141 of the drive element 12 in this embodiment are of a non-circular shape. In the locked position and in the intermediate position, the head does not fit through the orifice, whereas it fits through the orifice in the released position.

When the housing parts are pushed towards one another, the release element can be moved from its locked position into an intermediate position in which it holds the drive element locked and the trigger element is capable of moving the release element out of the intermediate position into the released position.

In view of the fact that neither the movement of the housing parts nor activation of the trigger element alone can lead to an injection being triggered, the system is particularly safe in terms of preventing unintentional triggering. In addition, this solution ensures that the automatic injector has to be placed on the skin of a patient before the trigger element can be activated.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising a housing formed by two housing parts moveable in relation to each other, a drive element urged by a spring in an injection direction and releaseably held against movement, and a release element moveable from a lock position to an intermediate position due to the movement of the housing parts, the drive element remaining held against movement when the release element is in the intermediate position, the release element further moveable from the intermediate position to a release position in which the drive element is released.

2. An automatic injector comprising an elongate housing comprising at least two housing parts which can be moved relative to one another, a drive element biased in an injection direction by at least one spring, a release element moveable from a locked position in which the drive element is locked into a release position in which the drive element is unlocked, and a trigger element, wherein a relative movement of the housing parts causes the release element to move from the locked position to an intermediate position in which the drive element is locked, actuation of the trigger element causing the release element to move from the intermediate position to the release position.

3. The automatic injector as claimed in claim 2, wherein the trigger element is moveable in a plane disposed transversely to a longitudinal axis of the automatic injector and the release element is moveable generally parallel to the longitudinal axis.

4. The automatic injector as claimed in claim 3, wherein the trigger element comprises a guide mechanism for guiding the release element during the movement from the intermediate position to the release position.

5. The automatic injector as claimed in claim 2, wherein the release element co-operates with a locking mechanism to lock the drive element in a biased position.

6. The automatic injector as claimed in claim 5, wherein the locking mechanism is moveable in a plane disposed transversely to the longitudinal axis of the automatic injector for unlocking purposes.

7. The automatic injector as claimed in claim 2, wherein the trigger element is moveable generally parallel to a longitudinal axis of the automatic injector and the release element is moveable a plane disposed transversely to the longitudinal axis.

8. The automatic injector as claimed in claim 2, wherein, in the locked position, the release element prevents the trigger element from moving.

9. The automatic injector as claimed in claim 2, the release element defining an orifice with a narrow portion, an annular groove provided on the drive element for engaging the narrow portion when the release element is in the locked position.

10. The automatic injector as claimed in claim 2, wherein the release element is guided so that it effects a rotating movement from the locked position into the release position.

11. The automatic injector as claimed in claim 10, wherein the release element defines a non-circular orifice and the drive element comprises a head having a non-circular cross-section which only fits through the orifice when the release element is in the release position.

12. A trigger-activatable injection device comprising:
a housing portion,
a reservoir for an injectable product,
an injection needle with a needle tip at one end,
a needle guard moveable relative to the housing portion and to the injection needle from a protective position generally surrounding the needle to a retracted position,
a drive mechanism for powering an injection, the drive mechanism releaseably held in a retaining engagement to prevent a driving movement,
a switching element coupled to the needle guard so that the movement of the needle guard to the retracted position causes the switching element to move from a locked position to a coupled position, and
an activatable trigger element for triggering the drive mechanism which, upon activation, is coupled with the switching element when the switching element assumes the coupled position and causes a movement of the switching element from the coupled position to a release position, wherein the retaining engagement of the drive mechanism is not released until the switching element is in the release position.

13. An injection device comprising:
a) a housing portion,
b) a reservoir containing a product,
c) an injection needle with a needle tip at a distal end,
d) a needle guard which is displaceable relative to the housing portion and to the injection needle from a protective position in which it surrounds the injection needle as far as beyond the needle tip, in the proximal direction as far as a retracted position,
e) a drive mechanism for an injection, which is locked in a releasable retaining engagement to prevent a driving movement,
f) a switching element, which is coupled with the needle guard so that the movement of the needle guard in the proximal direction causes the switching element to move from a locked position into a coupled position, and
g) an activatable trigger element for triggering the drive mechanism which, upon activation, is coupled with the switching element when the switching element assumes the coupled position and causes a movement of the switching element out of the coupled position into a released position, wherein
h) the retaining engagement of the drive mechanism is not released until the switching element is in the released position.

14. The injection device as claimed in claim 13, wherein the movement of the switching element from the locked position to the released position takes place in the same direction.

15. The injection device as claimed in claims 13, wherein the switching element is moved from the locked position to the released position by a movement in the proximal direction.

16. The injection device as claimed in claim 13, wherein the switching element is linearly guided in an axial direction from the locked position to the released position.

17. The injection device as claimed in claim 13, wherein the trigger element is displaceable transversely with respect to a direction in which the switching element is moveable to the released position.

18. The injection device as claimed in claim 13, wherein the trigger element moves the switching element from the coupled position to the released position by sliding on the switching element.

19. The injection device as claimed in claim 13, wherein at least one of the trigger element and switching element is provided with a switching cam by which it slides on the other in the coupled engagement, the switching cam inclined at an angle with respect to a direction in which the switching element is moved from the coupled position to the released position and with respect to a direction in which the trigger element is moved to the coupled engagement when activated.

20. The injection device as claimed in claim 13, wherein at least one blocking element holds the drive mechanism and the housing portion in a retaining engagement and the switching element holds the blocking element in the retaining engagement against an elastic force.

21. The injection device as claimed in claim 20, wherein the switching element pushes the blocking element into the retaining engagement against the elastic force.

22. The injection device as claimed in claim 21, wherein, when the switching element is in the released position, the blocking element is relieved of pressure by the switching element at least to the degree that the blocking element is able to move out of the retaining engagement.

23. The injection device as claimed in claim 21, wherein, when the switching element is in the released position, the blocking element is relieved of pressure by the switching element at least to the degree that the blocking element automatically snaps out of the retaining engagement.

24. The injection device as claimed in claim 22, wherein the switching element comprises at least one recess into which the blocking element snaps when the switching element is in the released position, thereby releasing the retaining engagement.

25. The injection device as claimed in claim 22, wherein the blocking element moves out of the retaining engagement transversely to the longitudinal axis of the injection needle.

26. The injection device as claimed in claim 22, wherein the switching element comprises a sleeve portion and pushes the blocking element into the retaining engagement by an internal surface of the sleeve portion, the internal surface defining a recess into which the blocking element snaps out of the retaining engagement.

27. The injection device as claimed in claim 13, wherein the needle guard drives the switching element from the locked position into the coupled position as it moves into the retracted position.

28. The injection device as claimed in claim 27, wherein the needle guard drives the switching element due to a pressure contact.

29. The injection device as claimed in claim 13, wherein the switching element is released from the needle guard as the switching element moves from the coupled position into the released position.

30. The injection device as claimed in claim 13, wherein the drive mechanism comprises an injection drive and a forward drive structure mounted to be displaceable toward the distal end in a forward drive direction relative to the housing portion and releaseably locked in the retaining engagement against a driving force of the injection drive, and wherein when the retaining engagement is released, the injection drive and the forward drive structure drive the injection needle in the forward drive direction relative to the needle guard.

31. The injection device as claimed in claim 30, wherein the drive mechanism has a drive element acting on the product in the reservoir, a dispensing structure and a dispensing drive which drives the drive element via the dispensing structure, and wherein the forward drive structure accommodates the dispensing structure and guides it in the forward drive direction.

32. The injection device as claimed in claim 30, wherein the drive mechanism has a drive element acting on the product in the reservoir, a dispensing structure and a dispensing drive which drives the drive element via the dispensing structure, and wherein the forward drive structure accommodates the dispensing structure and guides it in the forward drive direction, wherein the dispensing drive is formed by the injection drive.
